(19) United States
(12) Patent Application Publication
(10) Pub. No.: US 2004/0215318 A1
(43) Pub. Date: Oct. 28, 2004

(54) Timed delivery of therapeutics to blood vessels

(76) Inventor: Brian Kwitkin, Pembroke Pines, FL (US)

Correspondence Address: MEDTRONIC VASCULAR, INC., IP LEGAL DEPARTMENT, 3576 UNOCAL PLACE, SANTA ROSA, CA, 95403, US

(21) Appl. No.: 10/422,507

(22) Filed: Apr. 24, 2003

(51) Int. Cl. A61F 2/06

(52) U.S. Cl. 623/1.13; 623/1.42

(57) ABSTRACT

A method and apparatus for an intervention device for the surgical repair of aneurysm includes therewith a therapeutic delivery vehicle. The therapeutic delivery vehicle provides a time release of therapeutic agents, such as doxycycline, to the aneurysm site to reduce the presence of elastin attacking proteins in that location. Time release is affected by encasing the therapeutic agent in a time delivery vehicle. The time delivery vehicle may be diffusion based, or may introduce the therapeutic by both physical breakdown and diffusion mechanisms. The time delivery vehicle is further encapsulated in a porous membrane, such that materials in the pouch above a selected size remain within the porous membrane as the therapeutic agent is dispensed from the time delivery vehicle, but the therapeutic agent can pass through the pores to reach the aneurysmal site.

### BACKGROUND OF THE INVENTION

1. Field of the Invention

Embodiments of the present invention generally relate to the field of the treatment of body lumens, more particularly to the field of the treatment of blood vessels, and more particularly to the treatment of blood vessel aneurysms with stents, lined stents such as stent grafts, and the use of pharmaceutical agents therewith for the treatment of localized, blood vessel phenomena, such as aneurysm.

2. Description of the Related Art

Aneurysm, i.e., the enlargement of a blood vessel at a specific location therein to the point where rupture of the blood vessel is imminent, has been treated in the past by surgical intervention techniques, whereby the affected portion of the blood vessel is removed, or bypassed, so that the flow lumen is replaced by a synthetic graft. This treatment regimen is highly invasive for the patient undergoing it, and typically requires a multiple day post-operative hospital stay, as well as several months of recovery time until the patient has fully recovered from the surgery. Additionally, some patients may not capable of undergoing such a procedure.

To address the limitations imposed by surgical intervention to replace the aneurysmal blood vessel region with an artificial graft, a technique has been developed by which the aneurysmal blood vessel site been treated by placing what is known in the art as a stent graft, within the blood vessel in a position by which the tubular body of the stent graft spans the interior of the weakened area of the blood vessel wall. The stent graft, properly positioned, will allow blood to flow through the hollow tubular interior thereof, and also prevent blood, under systemic pressure, from reaching the weakened blood vessel wall at the aneurysmal site spanned by the tubular body thereof. However, there is still the opportunity for blood to reach the weakened wall location, such as through leakage of blood between the seal at the end of the stent graft and the vessel wall and thus into the weakened region, diffusion of blood through the graft material itself, or re-supply of blood into the aneurysmal sac from adjacent blood vessels. In each case, there is a renewed risk that the blood vessel may rupture. Furthermore, there remains a risk of additional deterioration of the blood vessel wall at the aneurysmal location even in the absence of blood leakage into the region isolated by the stent graft or the renewed supply of blood to the isolated region.

Typically, surgical intervention for aneurysm repair is not indicated until the blood vessel diameter, at the aneurysmal site, is 150 to 200% of its normal diameter. Below this threshold, the normal course of treatment has been to monitor the site, and if the diameter of the blood vessel wall at the aneurysmal site continues to expand beyond an undesirable threshold diameter, intervene surgically. Recently, it has been found that the application of certain antibiotics, such as doxycycline, can reduce the severity and/or progression of an aneurysm, and thereby reduce the likelihood of the need for surgical intervention to repair the aneurysm. It is postulated that the antibiotic reduces the level of an elastin attacking protein in the bloodstream and blood vessel wall, thereby reducing the severity of protein based attack on the elastin cells in the blood vessel wall and thus reduces the severity and the progression of the aneurysm. Typical antibiotic treatment requires the use of systemic antibiotics, either orally, intramuscularly or intravenously introduced, in a dosage sufficient to ensure that the quantity of antibiotic reaching the aneurysm is sufficient to affect the elastin attacking protein level at the aneurysm site. Thus, far more antibiotic must be used than that needed to treat the aneurysm, because a substantial portion of the antibiotic is directed by the blood stream to locations other than the aneurysmal site. The systemic use of antibiotics to treat localized treatment sites can lead to serious side effects, including the occurrence of drug resistant bacteria, gastrointestinal disruption, and the like. The longer the course of antibiotics is taken, and the higher the dosage, the higher the risk of serious side effects.

One additional proposed mechanism for treating blood vessels which are in an aneurysmal state, but for which surgery is not yet indicated, is to introduce “micro-spheres” containing a quantity of the therapeutic agent such as doxycycline, into the blood stream. Such microspheres are constructed to provide a time release of the pharmaceutical agent, and thus provide long term dosing of the aneurysmal site. These microspheres are typically configured to have a diameter on the order of at least 50 microns, such that sufficient therapeutic agent can be carried therein to enable a relatively long-term release of the therapeutic agent from the microsphere and into the bloodstream. However, microspheres of this size can cause substantial complications, such as the blockage of smaller capillaries or distal thrombosis. Additionally, only a small portion of the therapeutic agent released from the microspheres actually reaches the aneurysm, because the majority of the agent becomes distributed throughout the body by the patients' blood. Therefore, although the microspheres provide the patient with longer term regular dosing of the therapeutic agent, and thus free the patient from the need to regularly ingest or inject the agent, they do not eliminate the issue of the need for excess agent to treat a small locale, and the unintended consequences which may arise as a result.

Although the intravenous introduction of microspheres has been used to treat aneurysm, the intravenous use of such microspheres to treat the aneurysmal site after the placement of a stent graft therein is not possible, because the body of the stent graft will seal off the aneurysmal portion of the blood vessel wall from the microspheres, thereby preventing therapeutic delivery to the aneurysmal site. However, there still exists a need, post stent graft placement, to treat the aneurysmal site with therapeutic agents such as doxycycline, so as to reduce the severity and/or the progression of the aneurysm and thereby reduce the risk of aneurysm rupture, tear or other failure.

Therefore, there exists a need in the art for a localized drug delivery system, which will allow timed delivery of therapeutic agents to an aneurysmal site in a blood vessel, after placement of a bypassing element or prosthesis, such as placing a stent graft in the blood vessel to span the aneurysmal site, without the need for systemic application of the therapeutic agent.

### SUMMARY OF THE INVENTION

The present invention generally concerns methods and apparatus for the localized application of pharmaceutical and therapeutic agents. In one embodiment, the invention includes an encapsulation member, within which is provided a time release carrier containing, and capable of dosing over time, a therapeutic agent. In one embodiment, the encapsulation member is a pouch which is attached to the outer, i.e., blood vessel wall side, of a stent graft passing through an aneurysmal blood vessel, the stent graft thus isolating the aneurysmal region of the blood vessel from blood flow through the blood vessel and the pouch enabling delivery of the pharmaceutical agent to the aneurysmal blood vessel site. The encapsulation member is placed, by the method of locating a stent, stent graft, or other intervention device for spanning an aneurysm site through the interior of a blood vessel, and including the pouch on the exterior of the intervention device such that the pouch is positioned to release therapeutic agents into the space between the intervention device and the wall of the aneurysmal blood vessel.

The encapsulation member preferably is a pouch, constructed of a porous, biocompatible material, within which the therapeutic agent is located within a time-release carrier, such as microspheres. The pore size of the pouch material is less than that of the microsphere diameter, such that the microspheres remain encapsulated within the pouch as they release their therapeutic agent. Where the microspheres are degradable, the pores prevent release of the microsphere material from the pouch until they are of a sufficiently small size that their presence in the bloodstream will not result in systemic complications.

Preferably, the encapsulation member is attached to the stent graft at the proximal end thereof, i.e., at the end thereof into which blood flow enters the inner diameter of the stent graft to bypass the aneurysmal wall, and thus, as the stent graft is placed, any blood flow there past may come into contact with the aneurysmal blood vessel wall location. After the stent graft is in place, the encapsulation member will be surrounded with fluid in the space between the stent graft and the wall of the aneurysmal blood vessel, and thus the therapeutic agent will disperse through the fluid to provide the therapeutic agent to the wall of the aneurysmal location and treat the aneurysm to reduce the extension of the blood vessel wall and thereby reduce likelihood of rupture thereof.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a schematic view of a human, showing an aneurysmal aorta therein;

FIG. 2 is a sectional view of the aorta shown in FIG. 1, showing a repair vehicle, specifically shown a stent graft, therein;

FIG. 3 is an enlarged view of a portion of the stent graft of FIG. 2; and

FIG. 4 is an enlarged view, partially in section, of an encapsulation member used in conjunction with the repair vehicle of FIG. 2.

### DETAILED DESCRIPTION

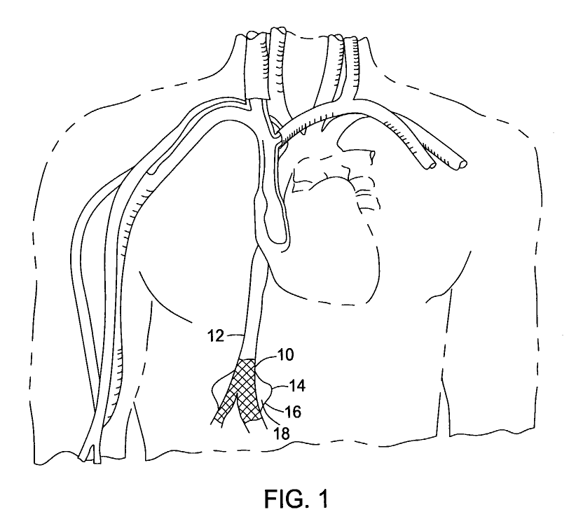

Referring initially to FIG. 1, there is shown an intravascular repair vehicle, specifically a stent graft 10, positioned in a blood vessel, in this embodiment, an aorta 12, and spanning, within the aorta 12, an aneurysmal portion 14 of the aorta 12. The aneurysmal portion 14 is formed of a bulging of the aorta wall 16, in a location where the strength and resiliency or the aorta wall 16 is weakened. As a result, an aneurysmal sac 18 is formed of distended vessel wall tissue. The stent graft 10 is positioned spanning the sac 18 and thereby provide both a secure passageway for blood flow through the aorta 12 and to seal off the aneurysmal portion 14 of the aorta 12 from additional blood flow from the aorta 12. The placement of the stent graft in the aorta 12 is a technique well known to those skilled in the art, and essentially includes the opening of a blood vessel in the leg, and the insertion of the stent graft 10 contained in a catheter into the vessel and through the vessel until deployed to be located in a spanning position across the aneurysmal portion 14 of aorta 12. The bifurcated stent graft 10 has a pair of branched sections bifurcating from a trunk portion thereof. This style of stent graft 10 is typically positioned in place by first inserting a catheter with the trunk portion into place through an artery in one leg, providing a first branched section to the aneurysmal location through the same artery and attaching it to the trunk portion at the aneurysmal site, and then inserting a catheter with the second branched section into place through an artery in the other leg of the patient, positioning it adjacent to the trunk portion, and likewise connecting it thereto. The procedure, and attachment mechanisms for assembling the stent graft in place in this configuration, is well known in the art, and is also disclosed in U.S. Pat. No. 6,203,568, incorporated herein by reference.

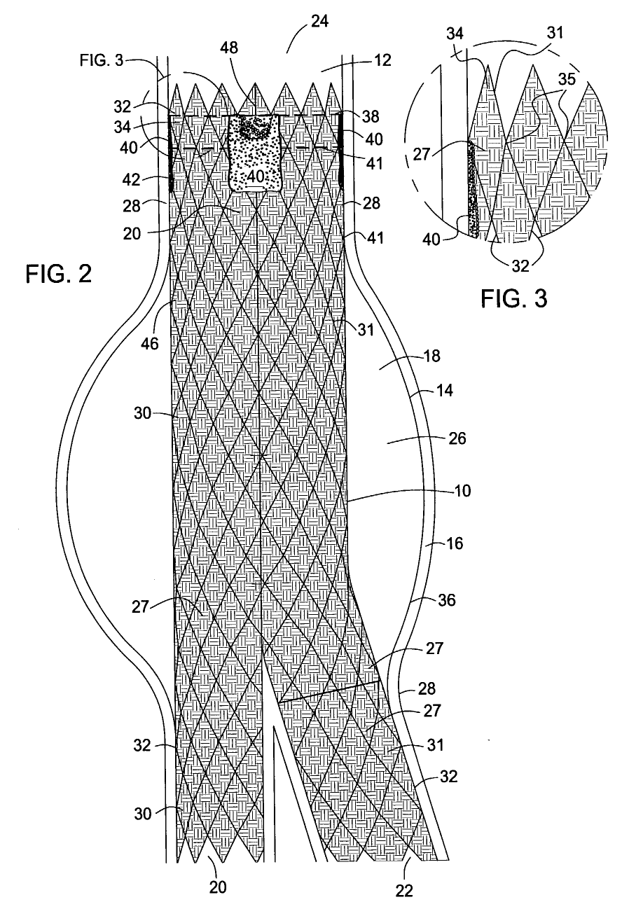

Referring now to FIG. 2, a bifurcated stent graft 10 is shown, being configured as a generally tubular member having distal ends 20, 22, a proximal end 24 and a cylindrical body portion 26. Although the bifurcated stent graft 10 is shown, in FIG. 2, in its fully assembled and positioned state, it is to be understood that the bifurcated stent graft 10 typically comprises at least three sections, a trunk portion 28, located in the lower portion of the descending aorta, and two minor diameter leg portions 30, 32, constructed integrally as one piece or with one leg joined thereto as shown. In one embodiment, the bifurcated stent graft 10 is configured such that each portion 28, 30 and 32 thereof includes a liner 27 externally supported by a tubular metal web 31 that expands to a pre-established diameter when placed in the aorta 12.

When assembled in place, the entire stent graft 10 spans the aneurysmal portion 14 of the aorta 12, including the sac 18, to seal such portion of the aorta 12 from blood flowing through the aorta 12. The metal web 31 includes a plurality of ring frame members each of which preferably includes a plurality of diamond shaped elements 34, typically provided as discrete lengths of diamond outlines such that a single length of such material can span the circumference of the stent graft 10 at the particular location where the hoop forms a portion of the metal web 31, and such diamond outlines are interconnected to form a continuous cylinder resulting in continuous support frame within which the liner 27 is supported. The diamond shaped elements 34 are preferably interconnected, as shown in FIG. 3, at the interstices 35 thereof, such as by tying them, welding them, or otherwise attaching them to one another. The diamond shaped elements 34 can be understood to form a ring or hoop, such as proximal ring 38 shown as those spanned by a dashed line in FIG. 2 formed of the extension of the diamond shaped elements about the circumference of the stent graft at the proximal end 24 thereof. Additionally, a first ring 41 again shown as those spanned by a second dashed line, circumscribes the stent graft 10 at the next inwardly disposed set of diamond shaped elements 34 disposed on the stent graft 10. Within the metal web 31 is disposed and supported the liner 27, which is affixed to the metal web 31 by mechanisms such as weaving or braiding the liner 27 to the metal web 31, or by mechanisms such as heat welding, bonding, gluing or ultrasonic welding. A bifurcated stent graft 10 of this construction is further disclosed in U.S. Pat. No. 6,203,568, previously incorporated herein by reference. When positioned in place in a blood vessel such as aorta 12, the stent graft will be in intimate contact with the blood vessel wall 16 for a length sufficient to ensure that blood will not readily flow between the stent graft 10 and the blood vessel wall 16. Typically, the stent graft 10 is in intimate contact with the blood vessel wall over the span of several rings from each of proximal end 24 and distal ends 20, 22.

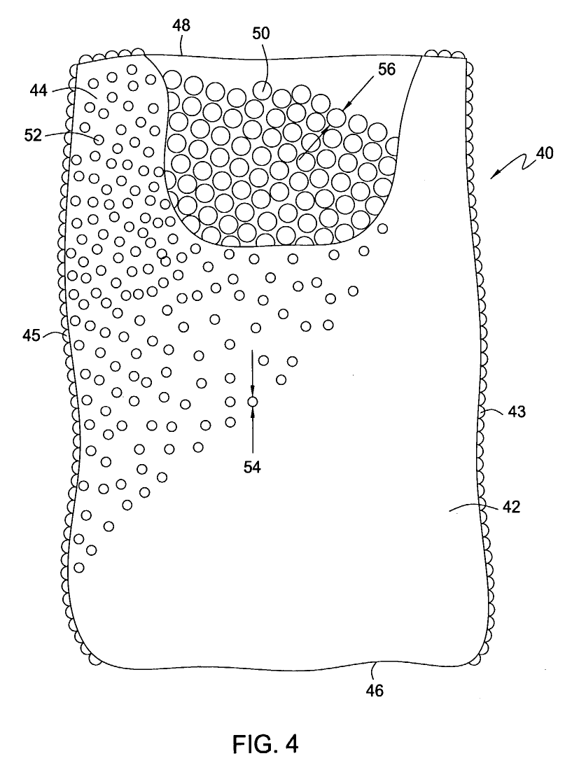

Referring still to FIG. 2, there is shown a pouch 40, connected to the proximal ring 38 of the bifurcated stent graft 10 on the outer surface thereof, i.e., positioned such that upon placement in an aneurysmal blood vessel location the pouch 40 is located between the stent graft 10 and the aneurysmal portion 14 of aorta 12. The pouch 40 is preferably positioned on the stent graft 10 by sewing it to proximal ring 38, thereby placing it in intimate contact with blood vessel wall 16. Pouch 40 includes a porous shell 42 having a plurality of pores 44 (Shown in FIG. 4) therein, and opposed sides 43, 45 and ends 46, 48 forming a generally rectangular pouch 40, end 48 being sewn to proximal ring 38 of trunk portion 28 of bifurcated stent graft 10.

Referring now to FIG. 4, pouch 40 is shown partially in cutaway, revealing a plurality of microspheres 50 packed therein, each of which preferably includes a pharmaceutical agent associated therewith. To maintain the microspheres 50 within the pouch 40, yet allow transport of fluids through the pouch 40, the porous shell 42 is manufactured from a porous biocompatible material having a plurality of pores 52 extending therethrough of a known diameter 54. Microspheres 50, when placed in the pouch 40, are sized to have a diameter 56 greater than the pore diameter 54. Preferably, pores 52 are on the order of five microns, and the microspheres 50 are initially ten to forty microns in diameter. Pouch 40 is preferably manufactured from a biocompatible material, such as Dacron, which is readily available with a pore size of approximately five microns. Pouch is preferably prepared by folding a sheet of the pouch material in half, and attaching together the opposed sides 43, 45 projecting from the crease occurring at the fold which forms end 46, such as by sewing, laser welding, adhesives or the like to leave an open end. The microspheres 50, or other pharmaceutical agent, is then loaded into the interior formed by securing the sides 43, 45, and the open end 48 is then sealed by similar means as those used to close the sides. The closure of the sides 43, 45 and open end 48 must ensure any remaining gaps between the folded over sheet at the seams are no greater than the pore diameter 52. Microspheres 50 for the present invention are preferably comprised of a biocompatible polymer, such as the copolymer, poly (DL-lactic-co-glycolic Acid), commonly known as PLGA, in which the pharmaceutical agent is encapsulated. When exposed to blood, the PLGA will break down into its co-constituents, thereby releasing the pharmaceutical agent trapped therein and thus releasing the agent within the pouch. The agent will then be dispersed from the pouch, either by virtue of diffusion processes, whereby the agent diffuses through the pores 52 of the pouch 40 and into contact with the adjacent blood vessel wall or blood or fluids thereadjacent, or by internal circulation of blood in the aneurysmal sac 18 causing blood or other fluids to flow through the pouch 40, and thereby be present in the region of the aneurysmal sac 18 of the aorta 12 to reduce the concentration of elastin attacking proteins adjacent the aneurysmal portion 14 of the blood vessel wall 16 and thus reduce the likelihood of progression of the aneurysmal condition.

The manufacture of the microspheres is accomplished by dissolving the copolymer, along with a desired quantity of the pharmaceutical agent, in a solvent such as an alcohol, and further adding water. The copolymer, combined with alcohol, water and pharmaceutical agent, forms an emulsion. This emulsion is heated, such as by placing the emulsion in a beaker, locating the beaker over a hot plate having a magnetically coupled stirring arrangement which couples to a stir rod in the beaker, and stirring the emulsion as the beaker and emulsion are heated to a temperature of the order of 70 to 80 degrees Celsius. As the solvents (alcohol and water) evaporate, the copolymer, having the pharmaceutical agent therein, precipitates out of solution as microspheres. Once a sufficient quantity of microspheres are precipitated, the emulsion is centrifuged, and the microspheres removed and dried. To provide microspheres having a certain diameter or desired range, the microspheres may be passed through filters or screens of known porosity, to separate microspheres into discrete groups of relatively equal size. Alternatively, the pharmaceutical agent can be encapsulated in a slab of the copolymer or other encapsulating material having time-release properties when exposed to blood or other body fluids. Such a slab is formed by dissolving copolymer in alcohol, along with the pharmaceutical agent, and then adding water and evaporating both the water and alcohol by heating the beaker or dish without stirring or agitating the beaker or dish in which the slab is prepared. The resultant slab is a matrix of copolymer having the pharmaceutical agent rapped therein, such that upon breakdown of the copolymer when exposed to blood, the pharmaceutical agent will be released. The resulting slab can be easily produced by those skilled in the art to have a thickness of between one-quarter to two and three quarter millimeters. Additionally, by modifying the mixture of the copolymers, as well as by the addition of plasticizers, and the like, the slab may be configured to have substantial flexibility, and thus be able to be twisted and bent when the stent graft 10 is placed in a catheter for delivery to the aneurysmal aorta 12. The slab is cut, after formation, to fit within pouch 40. The slab may, where sufficiently flexible, be cut to nearly the full size of the pouch 40, or alternatively be cut into strips, or smaller pieces, which are then stuffed into pouch 40. Preferably, the microspheres or slab are formed of PLGA, although other copolymers, such as PCL, are specifically contemplated.

After the pouch 40 is filled with the carrier, either the microspheres 50 or the slab, which are inserted into an open end 48 of the pouch 40, the end 48 is sown shut, and then attached to the stent graft 10, preferably at the proximal ring 38 or next adjacent ring thereof. Preferably, the pouch 40 has a width, i.e., a length spanning the circumferential direction of the stent graft when sewn thereto, on the order of five millimeters, a length, i.e., in the direction extending on the stent graft in the direction away from the distal end, of approximately 10 millimeters, and a thickness on the order of less than 3 mm.

The position of the pouch 40 adjacent the aneurysmal sac 18, and sealed from the blood passing through the aorta 12 by the stent graft 10, establishes the pouch 40 in a relatively sealed environment such that blood and other fluids in this region have a limited likelihood of being transmitted or passed from aneurysmal sac 18. Thus, the pharmaceutical agent will be released into the blood or other fluid in this relatively isolated region, such that a maximum concentration sustainable in the blood will likely be reached, after which no further pharmaceutical agent will enter the blood unless pharmaceutical agent already in the blood is dissipated, such as by reaction with elastin attacking proteins. As a result, substantial quantities of the pharmaceutical agent will remain in place for a longer period of time, increasing the time efficacy of the delivery system. Furthermore, by placing pouch 40 at the proximal end 24 of the stent graft 10, the pouch 40 will be positioned such that it is held in close contact with the blood vessel wall 14 at that location, and thus at least a portion of the therapeutic agent will be directly released from the pouch and into contact with the blood vessel wall 14 without the need to first pass through the blood or other fluid in the aneurysmal sac 18. Therefore the therapeutic agent can be directly delivered, without intervening diffusion of release into the blood or other fluid, thereby increasing its efficacy ion treating the aneurysmal site.

Preferably, multiple pouches 40 are used, each pouch being sewn at least one end thereof to the proximal or first ring of the stent graft 10, such that the spacing between adjacent pouches 40 extending about the circumference of the stent graft is relatively equal. Preferably at least four such pouches are equally spaced about the circumference of the stent graft 10 when placed across the aneurysmal aorta 12 (three shown in FIG. 2). Alternatively, multiple pouches 40 can be located both about the circumference of the stent graft 10, as well as longitudinally down its length.

Although the invention has been described herein in terms of using a specific degradable matrix element for time release of the pharmaceutical (or therapeutic) agent, the invention specifically contemplates use of other matrix/carrier materials, such as PCL, alginate, ceramics and inorganic polymers, which may or may not be degradable when exposed to blood. Where a non-degradable matrix is used, the rate of release of the pharmaceutical agent is diffusion limited, as blood or other liquid must enter the pores of the matrix and physically contact the agent therein to cause release into the blood or fluid.

Additionally, although the invention has been described in conjunction with the use of a stent graft to treat an aneurysmal aorta, the methods and apparatus herein are likewise applicable to treatment of other aneurysmal locations, as well as in conjunction with alternative repair vehicles. Finally, although the invention has been described in terms of using a time released pharmaceutical agent, the invention may also be practiced where other materials other than pharmaceuticals are used for time-release delivery. While the foregoing is directed to embodiments of the present invention, other and further embodiments of the invention may be devised without departing from the basic scope thereof.

1. A stent graft, comprising:
a tubular housing having an inner passageway therethrough and an outer, surface; and
at least one time-release delivery member provided on said outer surface.

2. The stent graft of claim 1, wherein said time-release delivery member includes at least one pouch having a pharmaceutical agent therein.

3. The stent graft of claim 2, wherein said pharmaceutical agent is encapsulated in a matrix located within said pouch.

4. The stent graft of claim 3, wherein said matrix, when exposed to blood, breaks down and thereby exposes pharmaceutical agents therein, to the blood.

5. The stent graft of claim 4, wherein said matrix includes PLGA.

6. The stent graft of claim 5, wherein the pharmaceutical agent is doxycycline.

7. The stent graft of claim 1, further including a plurality of pouches spaced about the outer surface.

8. The stent graft of claim 1, further including a web and a liner.

9. The stent graft of claim 8, wherein said pouch is sewn to said liner.

10. An intravascular repair vehicle for spanning a vascular defect and thereby substantially isolating such vascular defect from the conditions existing in the vascular vessel, comprising:
a tubular element having a generally hollow interior and an outer surface; including,
a time-release therapy element on the outer surface thereof.

11. The intravascular repair vehicle element of claim 10, wherein said time-release therapy element includes a pouch having the therapy element releasable maintained therein.

12. The intravascular repair vehicle of claim 11, wherein said therapy element is releasably maintained in a degradable polymer located within said pouch.

13. The intravascular repair vehicle of claim 12, wherein the polymer is PLGC.

14. The intravascular repair vehicle of claim 13, wherein the polymer is configured as a plurality of microspheres.

15. The intravascular repair vehicle of claim 13, wherein the polymer is configured as a slab of polymer containing said therapy element trapped therein.

16. The intravascular repair vehicle of claim 10, wherein said tubular element is a stent graft, and said therapy element is doxycycline.

17. The intravascular repair vehicle, wherein said therapy element is positioned on said tubular element such that, when said tubular element is positioned in an intravascular location, said therapy element is positioned in intimate contact with the vascular wall of the vascular vessel.

18. A method of providing a therapeutic agent to an intravascular site at which intervention to span a defective region of a vessel is contemplated, comprising the steps of;
providing an intravascular repair vehicle; and
providing a therapy element therewith in a position such that, upon placement of the repair vehicle in a vascular location, the therapy element will be positioned between the repair vehicle and the vessel wall and the therapy element will dispense a therapeutic agent over an extended period of time.

19. The method of claim 18, further including the steps of:
providing a degradable matrix with the therapy element;
encapsulating the therapeutic agent in the matrix.

20. The method of claim 19, wherein the degradable matrix comprises PLGA.

21. The method of claim 18, wherein the therapy element is a porous pouch having a therapeutic agent releasable maintained therein.

22. The method of claim 21. wherein the repair vehicle is a tubular member having an outer circumferential surface, and the therapy element is attached to an outer surface of the tubular member.

23. The method of claim 22, wherein the therapeutic agent is doxycycline, and
the vascular defect is an aneurysm.

24. The method of claim 23, wherein the vascular location at which the repair vehicle is to be placed is an aorta, and the repair vehicle is a stent graft.

25. The method of claim 18, further including the step of providing the therapy element in intimate contact with the wall of the vascular site being treated.
